(19) United States
(12) Patent Application Publication
(10) Pub. No.: US 2025/0120639 A1
(43) Pub. Date: Apr. 17, 2025

(54) DETECTING MEDICAL DEVICE ACCESSORY READINESS

(71) Applicant: Stryker Corporation, Portage, MI (US)

(72) Inventors: Rose Tingwei Yin, Bellevue, WA (US); Nattapon Chaimanonart, Lake Oswego, OR (US)

(73) Assignee: Stryker Corporation, Portage, MI (US)

(21) Appl. No.: 18/912,421

(22) Filed: Oct. 10, 2024

Related U.S. Application Data
(60) Provisional application No. 63/544,130, filed on Oct. 13, 2023.

(51) Int. Cl. A61B 5/28

(52) U.S. Cl. CPC A61B 5/28

(57) ABSTRACT

Techniques for detecting the readiness of an accessory device for use with a medical device are described. An example method includes detecting a capacitance of a capacitor including an electrode, a gel disposed on the electrode, and a conductive trace embedded in a plug. The plug is configured to couple to a medical device. A readiness of a medical device accessory is determined by analyzing the capacitance. The example method further includes outputting an indication of the readiness.

### CROSS-REFERENCE TO RELATED APPLICATION

This application claims priority to U.S. Provisional App. No. 63/544,130, which was filed on Oct. 13, 2023, is titled “DETECTING MEDICAL DEVICE ACCESSORY READINESS,” and is incorporated by reference herein in its entirety.

### BACKGROUND

Medical devices can be used to monitor patients, to administer treatments to patients, or both. Various medical devices include reusable components, such as electronics, processors, displays, and the like. However, some medical devices also utilize disposable components. For instance, a defibrillator is utilized with a disposable accessory device that includes electrode pads and a connector configured to plug into the defibrillator. A disposable accessory device for a medical device may be stored for an extended period of time in a packaged form. A user, for instance, is responsible for unpackaging the disposable accessory device and connecting it to the medical device.

### BRIEF DESCRIPTION OF THE DRAWINGS

FIG. 1 illustrates an example environment for checking the readiness of accessory devices.

FIGS. 2A to 2C illustrate steps of preparing a medical device for use. FIG. 2A illustrates a package disposed inside of the medical device. FIG. 2B illustrates an example of the package disposed in the medical device, wherein at least one electrode pad has been removed from a tray. FIG. 2C illustrates an example of one of the electrode pads being removed from the tray 206.

FIGS. 3A and 3B illustrate a capacitor indicative of a readiness status of an accessory device. FIG. 3A illustrates the capacitor in a first state. FIG. 3B illustrates the capacitor in a second state.

FIG. 4 illustrates a specific example of electrode pads packaged with a plug that enables self-testing.

FIGS. 5A and 5B illustrate examples of plugs of accessory devices that can be identified using an external device. FIG. 5A illustrates an example of a plug that includes a memory chip storing an identifier of an accessory device including the plug and/or an expiration date of the accessory device. FIG. 5B illustrates an example of a plug that includes a memory chip as well as sensors for detecting the readiness of a pair of electrode pads packaged with the plug.

FIG. 6 illustrates examples of an electrode pad package that can report results of a self-test to an external device.

FIG. 7 illustrates an example of a plug that can detect peel direction of electrode pads that are electrically connected to the plug.

FIG. 8 illustrates a cross-section of an example electrode pad with a PCB plug that can be used to detect a readiness state of the electrode pad.

FIG. 9A illustrates an example process for determining a readiness of a medical device accessory.

FIG. 9B illustrates an example process for identifying a problem associated with a medical device accessory.

FIG. 10 illustrates an example of an external defibrillator configured to perform various functions described herein.

### DETAILED DESCRIPTION

Disposable accessory devices for reusable medical devices, such as disposable electrode pads for external defibrillators, are ubiquitous. However, many disposable accessory devices eventually expire. For example, if a sufficient amount of water evaporates from a hydrogel coating on an electrode pad, the electrode pad may be unable to accurately detect an electrocardiogram (ECG) of a patient or may be unable to safely or effectively administer an electrical shock (e.g., for the purposes of pacing or defibrillation). In an expired state, the accessory devices may be unable to accurately detect physiological parameters from patients, may be unable to adequately treat patients, or may otherwise malfunction. In emergency situations, such malfunctions can have serious consequences for patients.

Manufacturers, for instance, can predict an expiration date of an accessory device and convey the predicted expiration date to users. For example, the packaging on a disposable electrode pad may indicate an expected expiration date, as well as a warning to refrain from using the disposable electrode pad if the expiration date has passed. However, relying solely on expiration dates to avoid the use of expired medical device accessories has a number of problems. For instance, a user, who may unpackage an accessory device in an emergency scenario in which a patient needs immediate medical attention, may not have sufficient time or attention to read or notice the expiration date of the accessory device before use. Accordingly, a distracted user may nevertheless attempt to use the accessory device even if the expiration date has passed. Second, the expiration date may reflect an incorrect estimation of the usable lifetime of the accessory device. In some examples, the expiration date may be an underestimation or overestimation of the usable lifetime of the accessory device, due to the storage environment of the accessory device. For instance, if the accessory device was stored in a relatively hot environment, it may lose readiness before its printed expiration date. In some cases, usable accessory devices may be unnecessarily discarded, or unusable accessory devices may be used, if users rely solely on expiration dates for determining accessory device readiness.

Various implementations described herein address these and other problems by specifically detecting the readiness of an accessory device, such as prior to use with a medical device. In some implementations, the accessory device may detect its own readiness before it is unpackaged. Example accessory devices described herein include sensors that detect an electrical characteristic indicative of their readiness. For instance, an example electrode pad includes a capacitive sensor that detects whether a gel of the electrode pad is unsuitable for use. In some cases, sensors are integrated into a connector (e.g., a plug) of example accessory devices. According to some implementations, sensors are powered by power sources that are physically integrated into the accessory devices. In some examples, sensors are powered wirelessly. According to some cases, accessory devices include memory storing readiness information, such as expected expiration dates, identifiers, accessory device types, and the like. In various examples, accessory devices can detect and report their readiness statuses prior to being physically coupled to external devices, such as defibrillators or other standalone medical devices.

According to some implementations, accessory devices detect whether they are unpackaged in such a way that would potentially interfere with their function. For instance, example electrode pads include multiple sensors that detect times (e.g., a timing sequence) at which portions of the electrode pads are peeled away from a substrate. Based on the times, the electrode pads detect their peeling directions. Accordingly, example accessory devices described herein are configured to detect and report whether they have been unpackaged correctly or incorrectly.

In some cases, sensors in example accessory devices described herein are utilized when the accessory devices are physically coupled to standalone medical devices. For example, sensors integrated into accessory devices may be powered by medical devices connected to the accessory devices. In some examples, medical devices connected to the accessory devices may identify and report the readiness statuses of the accessory devices to which they are connected.

Implementations of the present disclosure will now be described with reference to the accompany figures.

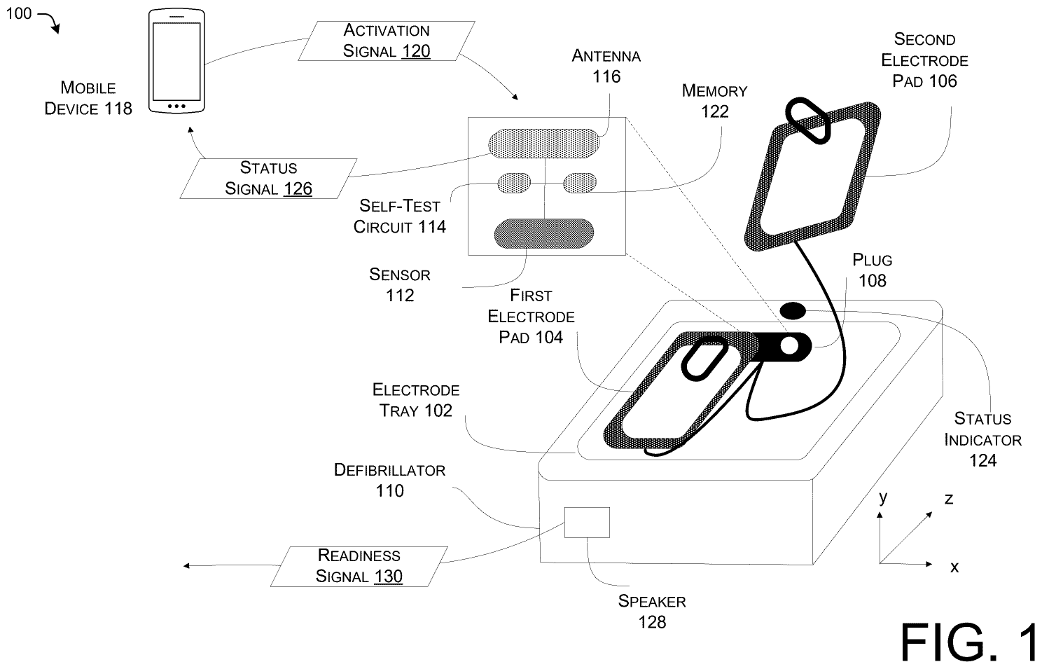

FIG. 1 illustrates an example environment 100 for checking the readiness of accessory devices. As used herein, the term “readiness,” and its equivalents, may refer to a physical characteristic of an accessory device indicative of whether the accessory device is likely to function in an expected manner. For instance, an accessory device that has greater than a threshold likelihood to accurately detect a physiological parameter, to administer a therapy (e.g., an electrical shock), or to otherwise adequately engage in a function it is designed to perform would be designed as “ready” or as having an “adequate readiness state.” As used herein, the term “accessory device,” and its equivalents, refers to an apparatus that enables a standalone medical device to detect a physiological parameter of a subject and/or to administer a therapy to the subject. Examples of accessory devices include electrode pads, blood pressure cuffs, airway tubes (e.g., intubation tubes), capnography sensors, oximeters (e.g., pulse oximeters), thermometers, catheters, flow sensors, and so on. In various examples, accessory devices are disposable devices.

As used herein, the terms “standalone medical device,” “medical device,” and their equivalents, can refer to an apparatus that utilizes an accessory device to monitor or to administer a treatment to a patient. In many cases, a standalone medical device is self-powered (e.g., via a battery or a means to connect to mains current), includes input devices (e.g., buttons, touchscreen, etc.) configured to receive user input signals, includes output devices (e.g., speakers, displays, etc.) configured to output signals to users, and includes processing capabilities (e.g., at least one processor) configured to analyze data, report patient conditions, recommend therapy administration, generate a therapy to be administered, or any combination thereof. Examples of standalone medical devices include monitor-defibrillators, automated external defibrillators (AEDs), ventilators, and patient monitors. In various examples, standalone medical devices are reusable.

As used herein, the term “physiological parameter,” and its equivalents, may refer to a metric indicative of a characteristic of a subject's body. Examples of physiological parameters include an ECG, an impedance (e.g., a transthoracic impedance), a blood pressure, a capnograph, an end-tidal CO₂, a photoplethysmograph, a blood oxygenation (e.g., SpO₂), blood flow (e.g., blood velocity), a respiration rate, a heart rate, a pulse rate, or any combination thereof. In some cases, physiological parameters include electrical signals detected via electrodes, such as an ECG, an electroencephalogram (EEG), an electrooculogram (EOG), or an electromyograph (EMG).

As used herein, the terms “treatment,” “therapy,” and their equivalents, may refer to a substance, force, or signal that can be administered to the subject for the purpose of resolving a pathology and/or reducing a symptom. Examples of treatments include the administration of one or more electrical shocks (e.g., defibrillation shocks, pace pulses, etc.) to resolve an arrhythmia. Other types of treatments include the administration of assisted ventilation, chest compressions, medications, and the like.

In particular, the environment 100 includes an electrode tray 102 that at least partially encloses a first electrode pad 104 and a second electrode pad 106. In some examples, the environment 100 includes an electrode pouch that encloses the first electrode pad 104 and the second electrode pad 106, wherein the electrode pouch is either stored inside of the electrode tray 102 or is used as an alternative to the electrode tray 102. The first electrode pad 104 and the second electrode pad 106, for instance, are configured to detect an electrical signal from a subject and/or to administer an electrical signal to the subject. In various cases, the first electrode pad 104 and the second electrode pad 106 are configured to be applied externally. For instance, the first electrode pad 104 and the second electrode pad 106 are configured to be disposed on skin of the subject.

In various cases, the electrode tray 102 includes a housing in which the first electrode pad 104 and the second electrode pad 106 are stored. Although not specifically illustrated in FIG. 1, the electrode tray 102 may be enclosed by a lid, which may be removed from the electrode tray 102 prior to use. The electrode tray 102 and the lid, for instance, may substantially protect the first electrode pad 104 and the second electrode pad 106 from dust, water, and other contaminants. In various cases, the electrode tray 102 and the lid may maintain an internal humidity that prevents evaporation of water from the first electrode pad 104 and the second electrode pad 106. For instance, the lid may form a fluid-tight seal with the electrode tray 102 prior to removal. According to some cases, a user may remove the lid by peeling the lid from the electrode tray 102. The electrode tray 102 and/or the lid may include a polymer, such as polystyrene. In some cases, the lid is adhered to the electrode tray 102 by a fluid-tight adhesive.

In various cases, the first electrode pad 104 and the second electrode pad 106 are configured to transmit an electrical signal to a subject (e.g., a patient) and/or to detect an electrical signal from the subject. According to various cases, each one of the first electrode pad 104 and the second electrode pad 106 may include an electrode. Each electrode, for instance, includes a conductive film. In some cases, each electrode includes silver/silver chloride (Ag/AgCl), titanium, copper, or any combination thereof. The first electrode pad 104 and the second electrode pad 106 may further include a substrate on which the electrode is disposed. The substrate, in various cases, is electrically insulative. According to some cases, the substrate includes an insulative polymer foam material. In various cases, a border of the substrate (e.g., around the conductive film) is covered with a biocompatible adhesive, such that each one of the first electrode pad 104 and the second electrode pad 106 may be adhered to the skin of the subject and each electrode may make electrical contact with the skin of the subject.

In some cases, each of the first electrode pad 104 and the second electrode pad 106 further includes a gel that is configured to enhance the electrical coupling between each electrode and the skin of the subject. The gel, for instance, is a hydrogel. In various cases, the gel includes one or more electrolytes. The gel is electrically conductive, for instance. When the first electrode pad 104 and the second electrode pad 106 are applied to the skin of the subject, the gel is disposed between the skin and the respective electrodes in the first electrode pad 104 and the second electrode pad 106.

The first electrode pad 104 and the second electrode pad 106 are connected to a plug 108, which is also at least partially enclosed by the electrode tray 102. In particular cases, a first wire extends between the electrode of the first electrode pad 104 and the plug 108, and a second wire extends between the electrode of the second electrode pad 106 and the plug 108. The plug 108 is an electrical connector configured to removably couple the electrode of the first electrode pad 104 and the electrode of the second electrode pad 106 to a standalone medical device, such as a defibrillator 110.

In various cases, the defibrillator 110 is configured to monitor and/or administer a therapy to a subject. In particular cases, the defibrillator 110 may be configured to detect one or more physiological parameters (e.g., ECG, transthoracic impedance, etc.) from the subject. The defibrillator 110, in some cases, is configured to analyze the physiological parameter(s) and determine if the subject is exhibiting a shockable heart rhythm (e.g., ventricular fibrillation (VF)). Further, the defibrillator 110 may output a therapy to the subject that treats the shockable heart rhythm. In particular cases, the defibrillator 110 outputs one or more electrical shocks that, when received by the heart of the subject, may cause the heart of the patient to resume a non-shockable heart rhythm, such as a normal sinus rhythm with defined QRS complexes. That is, the defibrillator 110 may be configured to defibrillate the subject.

According to some examples, the defibrillator 110 is an automated external defibrillator (AED) configured to be operated by a bystander, who may be someone without specialized training. For example, the defibrillator 110 may be stored in a cabinet that is located in a public area, such as a school, an airport, or an office. An individual in the public area may suddenly, and unexpectedly, experience a shockable heart rhythm. In various cases, the shockable heart rhythm may prevent the heart of the individual from effectively pumping blood to the individual's brain and other vital organs. If left untreated, the shockable heart rhythm may cause permanent damage to the individual's body, or may even lead to death. In particular, it is advantageous if the shockable heart rhythm is treated within minutes of initiating. Accordingly, the defibrillator 110 may be permanently stored in the public area, and can be used immediately by a bystander if the individual exhibits symptoms of the shockable heart rhythm in its vicinity.

The defibrillator 110, for example, may be configured to provide instructions to the bystander indicating how to utilize the defibrillator 110. In particular cases, the defibrillator 110 may output the instructions visually (e.g., on a screen or printed material on a housing of the defibrillator 110 itself), audibly (e.g., via audible instructions), or a combination thereof. The defibrillator 110, in various cases, instructs the bystander on how to remove the first electrode pad 104 and the second electrode pad 106 from the electrode tray 102 and how to apply the first electrode pad 104 and the second electrode pad 106 to the chest of the subject. The defibrillator 110 may automatically detect an ECG from the subject via the set of the first electrode pad 104 and the second electrode pad 106, and may also determine whether the ECG is indicative of the shockable rhythm by analyzing the ECG. If the defibrillator 110 detects the shockable rhythm, the defibrillator 110 may output an instruction to administer an electrical shock to the subject via the first electrode pad 104 and the second electrode pad 106. Thus, the function of the defibrillator 110, the analysis of the condition of the subject, and the treatment of the subject, are all dependent on the readiness of an accessory device that includes the first electrode pad 104, the second electrode pad 106, and the plug 108.

In the environment 100, the electrode tray 102 (e.g., with the lid attached) is disposed inside of the defibrillator 110. According to some cases, the electrode tray 102 is stored in the defibrillator 110 for an extended period of time prior to use. For instance, the electrode tray 102 is stored for days, weeks, months, or years prior to use.

However, due to a lengthy storage time, there is a possibility that the first electrode pad 104 and/or the second electrode pad 106 is unsuitable for use at the time an emergency occurs. For instance, due to the lengthy storage time, the gel on the first electrode pad 104 and/or the second electrode pad 106 may have dried or otherwise become degraded, which may reduce the efficacy of a defibrillation therapy administered by the defibrillator 110 using the first electrode pad 104 and the second electrode pad 106.

Other problems may also prevent the effective use of the first electrode pad 104 and the second electrode pad 106 to administer a therapy to a patient. In some cases, a rescuer is instructed to remove the first electrode pad 104 and the second electrode pad 106 from the electrode tray 102 by peeling the first electrode pad 104 and the second electrode pad 106 from the electrode tray 102. Once removed from the electrode tray 102, the rescuer my apply the first electrode pad 104 and the second electrode pad 106 to the patient, such as by adhering the first electrode pad 104 and the second electrode pad 106 to the chest of the patient. In particular examples, the peeling direction of the first electrode pad 104 and the second electrode pad 106 may impact their function. For instance, if the first electrode pad 104 and the second electrode pad 106 are removed from the electrode tray 102 via an incorrect peeling direction (e.g., a positive z direction), a component of the electrode tray 102 (e.g., a plastic cover) may be retained on surfaces of the first electrode pad 104 and the second electrode pad 106. This component, in some examples, may prevent the first electrode pad 104 and the second electrode pad 106 from making electrical contact with the chest of the patient. In contrast, if the first electrode pad 104 and the second electrode pad 106 are removed from the electrode tray 102 via a correct peeling direction (e.g., a negative z direction), the component of the electrode tray 102 is removed from surfaces of the first electrode pad 104 and the second electrode pad 106.

According to various implementations of the present disclosure, a readiness of the first electrode pad 104 and the second electrode pad 106 can be detected prior to use. In particular cases, the readiness of the electrode pad 104 and the second electrode pad 106 can be detected using a sensor 112 that is disposed in the electrode tray 102. For instance, the sensor 112 may be disposed inside of the plug 108 configured to connect the first electrode pad 104 and the second electrode pad 106 to the defibrillator 110.

In particular examples, the sensor 112 is configured to detect a capacitance of a capacitor that includes components of the first electrode pad 104, as well as a capacitor that includes components of the second electrode pad 106. For instance, the gel disposed on the first electrode pad 104 and the second electrode pad 106 includes a dielectric material. Thus, the sensor 112 may include a conductive material that serves as one plate of a capacitor, an electrode in the first electrode pad 104 or the second electrode pad 106 may serve as another plate of the capacitor, and the gel disposed on the first electrode pad 104 and the second electrode pad 106 may serve as the dielectric layer disposed between the plates. The capacitance of the capacitor can be represented by the following Equation 1:

$$C = {ε}_{0} {ε}_{r} \frac{A}{d} \qquad ( 1 )$$

wherein ε₀ is the electric constant, ε_{f} is the relative permittivity of the material(s) between the plates (e.g., including the gel), A is the area of overlap of the two plates, and dis the distance between the two plates. The capacitance of the capacitor is indicative of the condition and status of the first electrode pad 104 or the second electrode pad 106.

In various cases, the capacitance is indicative of a condition of the gel. As the gel ages, it may dry out and become less effective at promoting electrical contact between the chest of the patient and the first electrode pad 104 or the second electrode pad 106. The water content of the gel, in various implementations, also impacts its relative permittivity. In particular, the relative permittivity of the gel increases as the amount of water in the gel decreases. In various implementations, if the capacitance of the capacitor is greater than a threshold capacitance, the corresponding electrode may be determined to be expired or otherwise unsuitable for use by the defibrillator 110.

According to some implementations, multiple capacitors associated with the same electrode pad can be used to determine the peeling direction from which the electrode pad is removed from the electrode tray 102. For example, the sensor 112 may include at least two conductive materials that respectively form at least two capacitors with the electrode of the first electrode pad 104 or the second electrode pad 106 and the gel disposed on the electrode. The sensor 112 may detect the capacitances of the capacitors. As noted in Equation 1, the capacitance of a capacitor increases as the distance between the plates increases. Thus, by monitoring times (e.g., a timing sequence) at which capacitances of the respective capacitors increase suddenly (e.g., above a threshold capacitance), the sensor 112 may detect the direction at which the first electrode pad 104 or the second electrode pad 106 is removed from the electrode tray 102. In various implementations, an incorrect peeling direction can be identified prior to using the first electrode pad 104 or the second electrode pad 106 to treat the patient.

According to some examples, the readiness and/or peeling direction of the first electrode pad 104 or the second electrode pad 106 can be identified solely based on components within the accessory device itself, such as solely based on components within the electrode tray 102. For instance, the readiness and/or peeling direction can be detected before the accessory device is connected to the defibrillator 110, a battery, or some other active power source.

In various cases, the sensor 112 is electrically coupled to a self-test circuit 114. The self-test circuit 114, according to some examples, is disposed in the plug 108. The self-test circuit 114, in various cases, is configured to analyze the capacitance detected by the sensor 112. In some cases, the self-test circuit 114 includes analog and/or digital circuitry, such as an application-specific integrated circuit (ASIC) configured to perform various functions described herein. In some examples, the self-test circuit 114 includes a processor. For instance, the self-test circuit 114 may be configured to receive an indication of the capacitance(s) from the sensor 112, such as in the form of an electrical signal that is indicative of the capacitance(s). The self-test circuit 114, in some cases, is configured to compare the capacitance(s) to at least one threshold. Based on the comparison, the self-test circuit 114 may be configured to determine a readiness of the first electrode pad 104 or the second electrode pad 106, or may be able to detect whether the first electrode pad 104 or the second electrode pad 106 was peeled incorrectly from the electrode tray 102. In various cases, the self-test circuit 114 may output a signal indicative of the readiness or peel direction.

Various types of power may be supplied to the sensor 112 and the self-test circuit 114. According to some instances, the sensor 112 and self-test circuit 114 are electrically connected to a power source, such as a battery (not illustrated). For instance, the battery may be disposed in the plug 108.

In some examples, the self-test circuit 114 is electrically connected to an antenna 116 that is configured to supply power to the self-test circuit 114 and the sensor 112. In various cases, the antenna 116 is configured to receive an electromagnetic signal, wirelessly, from an external device. For example, the electromagnetic signal may have a frequency in a range of 10 to 2500 megahertz (MHZ). For instance, the electromagnetic signal is a BLUETOOTH or Near-Field Communication (NFC) signal. The electromagnetic signal may induce a current in the antenna 116, which may thereby supply energy to the sensor 112 and the self-test circuit 114 connected to the antenna 116. Accordingly, the sensor 112 and the self-test circuit 114, in some cases, is wirelessly powered by a device that is external to the accessory device.

In particular cases, a mobile device 118 is configured to transmit an activation signal 120 to the antenna 116 of the accessory device. The activation signal 120, for instance, is an electromagnetic signal that activates the sensor 112 and the self-test circuit 114. In various cases, the sensor 112 is configured to detect a capacitance in response to the antenna 116 receiving the activation signal 120.

According to some examples, the self-test circuit 114 is further electrically connected to memory 122. The memory 122, for instance, may store an identifier of the accessory device and/or an expected expiration date of the accessory device. In some examples, the self-test circuit 114 is configured to determine the readiness of the first electrode pad 104 or the second electrode pad in response to determining that the expiration date of the accessory date has passed. Accordingly, in some cases, implementations of the present disclosure may enable the use of accessory devices that are genuinely ready for use, but would otherwise be discarded due to expected expiration dates.

The accessory device may include one or more output devices configured to output an indication of the readiness, peeling direction, or other information related to the condition of the accessory device. For instance, a status indicator 124 may be disposed on the plug 108 or some other portion of the electrode tray 102. In various cases, the status indicator 124 is a light source, screen, or other type of output device. According to some examples, the status indicator 124 may output a signal (e.g., a visual signal) when the self-test circuit 114 detects a problem with the accessory device. That is, the self-test circuit 114 may cause the status indicator 124 to output the signal. For example, the status indicator 124 may be illuminated (e.g., using a red color) when the first electrode pad 104 or the second electrode pad 106 is expired or has been incorrectly peeled from the electrode tray 102. According to some cases, the status indicator 124 is powered by an internal power source (e.g., a battery) and/or is powered by the activation signal 120, which induces the current in the antenna 116.

According to some examples, the antenna 116 is further configured to transmit a status signal 126 to an external device, such as the mobile device 118. The status signal 126, in various implementations, is an electromagnetic signal that encodes data. For instance, the status signal 126 is a communication signal. The status signal 126, in various examples, indicates the readiness, peeling direction, or other information related to the condition of the accessory device. For example, the self-test circuit 114 may cause the antenna 116 to transmit the readiness signal 130.

In some cases, the readiness signal 130 identifies the accessory device itself. For example, the readiness signal 130 includes identifying information that indicates a type of the accessory device, a model number of the accessory device, a manufacturer of the accessory device, an expected expiration date of the accessory device, a type of medical device to be used with the accessory device, or any combination thereof. The identification information, in some cases, is stored in the memory 122. In some examples, the identification information is encoded in a shape of the antenna 116. For example, the antenna 116 may include a wireless transceiver (e.g., an NFC chip) that can be read by the mobile device 118 using the activation signal 120. Thus, the mobile device 118 may identify the accessory device in the electrode tray 102 based on the status signal 126.

In various implementations described herein, a readiness, status, or peeling direction of the accessory device can be identified before the accessory device is connected to a medical device, such as the defibrillator 110. However, implementations are not necessarily so limited.

In some cases, the defibrillator 110, at least partly, determines a readiness of the accessory device. For example, the plug 108 may be electrically connected to the defibrillator 110, such that a power source in the defibrillator 110 may provide electrical energy to the sensor 112, the self-test circuit 114, the antenna 116, the memory 122, the status indicator 124, or any combination thereof. For instance, the self-test circuit 114 may be activated when the plug 108 is connected to the defibrillator 110.

According to some implementations, the defibrillator 110 includes circuitry that detects other types of conditions of the accessory device. For example, the defibrillator 110 may include a circuit that, when the plug 108 is connected to a socket of the defibrillator 110, includes at least one of the capacitors described herein. For example, an electrical connection between the plug 108 and the defibrillator 110 completes a circuit that includes a power source in the defibrillator 110 and a capacitor including the electrode of the first electrode pad 104 or the electrode of the second electrode pad 106. The circuit may further include a sensor (not illustrated) that detects a current (or other type of electrical signal) through the circuit. When the plug 108 is connected to the defibrillator 110, the sensor may at least temporarily detect a nonzero current through the circuit. However, if the plug 108 is disconnected from the defibrillator 110, the circuit may be unable to detect a current through the circuit. In various implementations, the defibrillator 110 may therefore detect when the accessory device is connected, or if the accessory device is disconnected, from the defibrillator 110.

In various cases, the defibrillator 110 outputs a signal indicative of the status, readiness, or connection of the accessory device. For instance, the defibrillator 110 includes a speaker 128 that is configured to output a readiness signal 130. The speaker 128, for instance, audibly outputs the readiness signal 130 to a user (e.g., a rescuer, a person responsible for maintenance of the defibrillator 110, or the like). According to various cases, the readiness signal 130 indicates whether the first electrode pad 104 is expired or unexpired, whether the second electrode pad 106 is expired or unexpired, whether the first electrode pad 104 has been removed from the electrode tray 102 in a correct peeling direction, whether the second electrode pad 106 has been removed from the electrode tray 102 in a correct peeling direction, whether the plug 108 is connected to the defibrillator 110 (e.g., whether the plug 108 should be re-connected to the defibrillator 110), or any combination thereof. For example, the readiness signal 130 may include a recording of a voice reporting a readiness of the accessory device and/or the defibrillator 110.

In some cases, the defibrillator 110 outputs a communication signal indicative of the status, readiness, or connection of the accessory device. For example, the defibrillator 110 may include a transceiver (e.g., an antenna, not illustrated) that is configured to transmit a communication signal indicating information that could otherwise be included in the readiness signal. In some cases, the defibrillator 110 transmits the communication signal to an external device, such as the mobile device 118. Accordingly, the defibrillator 110 may report the status of the accessory device, or the connection between the accessory device and the defibrillator 110.

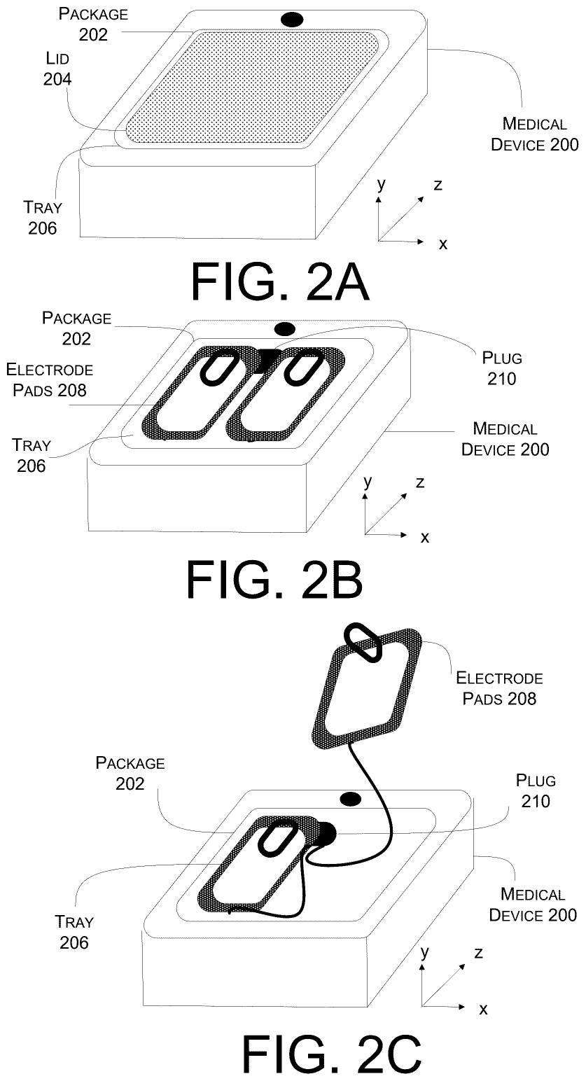

FIGS. 2A to 2C illustrate steps of preparing a medical device 200 for use. For instance, the medical device 200 is the defibrillator 110 described above with respect to FIG. 1.

FIG. 2A illustrates a package 202 disposed inside of the medical device 200. According to some examples, a housing of the medical device 200 is at least partially enclosed around a space.

The package 202, in various implementations, holds an accessory device. In various cases, the accessory device includes one or more electrode pads as well as a plug configured to electrically connect the accessory device to the medical device 200. During storage, the package 202 includes a lid 204 and a tray 206, wherein the lid 204 is adhered to a portion of the tray 206. In various cases, the lid 204 and the tray 206 are sealed together with a fluid-tight seal. Accordingly, the package 202 may be stored for an extended period of time (e.g., weeks, months, or years) in order to enable the readiness of the accessory device when a need arises.

In some implementations, the accessory device may self-report its readiness while still enclosed by the lid 204. For example, the plug of the accessory device may include a sensor configured to detect whether a gel disposed on one or both of the electrode pads is intact, thereby determining whether the electrode pads are ready for use or expired. In some cases, the sensor is a capacitive sensor. According to some examples, the accessory device may further include an antenna configured to receive an electromagnetic signal that it can use to power the sensor and/or to report the readiness of the accessory device.

FIG. 2B illustrates an example of the package 202 disposed in the medical device 200, wherein the lid 204 has been removed from the tray 206. For example, a user may have physically peeled the lid 204 away from the tray 206, thereby exposing the accessory device. In various cases, the accessory device includes a pair of electrode pads 208 as well as a plug 210. In various cases, the plug 210 is configured to connect the electrode pads 208 to the medical device 200.

In some implementations, the accessory device is configured to report its readiness before the plug 210 is connected to the medical device 200 or after the plug 210 is connected to the medical device 200. For example, a sensor in the plug 210 may determine the readiness of the accessory device. In some examples, the sensor is wirelessly powered by an antenna, such as an antenna located in the plug 210. In some instances, the sensor is powered by a power supply of the medical device 200, such as a capacitor, a battery, or the like. In various implementations, the accessory device itself (e.g., via the antenna or a transceiver) may report its readiness to an external device or to the medical device 200. In some implementations, the medical device 200 may report the readiness of the accessory device. For instance, the medical device 200 may include a transceiver that transmits, to an external device, a communication signal indicating the readiness of the electrode pads 208. In some cases, the accessory device or the medical device 200 includes an output device (e.g., a speaker, a display, a light source, etc.) that outputs a signal, to a user, indicating the readiness of the electrode pads 208.

FIG. 2C illustrates an example of one of the electrode pads 208 being removed from the tray 206. In various implementations, both of the electrode pads 208 are removed from the tray 206 before use. For instance, a user may place the electrode pads 208 on the skin of a patient, so that the patient can be monitored and/or treated by the medical device 200. The user, for instance, removes the electrode pads 208 by peeling the electrode pads 208 from the tray 206. In some cases, the electrode pads 208 are peeled away from a substrate, which may include paper, a polymer, or a combination thereof.

In various implementations, the electrode pads 208 are released from the substrate when the electrode pads 208 are peeled in a predetermined peeling direction. For example, the predetermined peeling direction may be a negative z direction, when the package 202 has the arrangement illustrated in FIG. 2C. However, in some cases, the electrode pads 208 are not released from the substrate when the electrode pads 208 are peeled from the tray 206 in another peeling direction (e.g., a positive z direction). If the substrate remains on surfaces of the electrode pads 208 when the user attempts to place the electrode pads 208 on the skin of the patient, the substrate may prevent the electrode pads 208 from receiving an accurate physiological signal from the patient and/or from outputting an appropriate therapy signal to the patient. In some cases, the electrode pads 208 are printed with instructions showing the appropriate peeling direction, such as arrows indicating the predetermined peeling direction. Further, the electrode pads 208 illustrated in FIGS. 2B and 2C have handles that facilitate the removal of the electrode pads 208 via the predetermined peeling direction. However, in some cases, the instructions and handles may be insufficient to guarantee that the electrode pads 208 are removed in the predetermined peeling direction.

According to some implementations, at least one sensor disposed in the plug 210 may be configured to detect the direction(s) from which the electrode pads 208 are removed from the tray 206. For example, the plug 210 may include a first sensor and a second sensor disposed underneath one of the electrode pads 208 in the y direction. The first sensor and the second sensor, for instance, may be separated from each other in the z direction. According to some examples, the first sensor and the second sensor may detect when a portion of the electrode pads 208 above the respective sensors is moved away from the sensors in a y direction. For instance, the first sensor and the second sensor may be capacitive sensors. Based on the detected separation of the portions of the electrode pads 208, the sensors may enable detection of the direction from which the electrode pads 208 are removed from the tray 206. According to some examples, the first and second sensors are powered by energy from an antenna receiving an electromagnetic signal from an external device. In some cases, the first and second sensors are powered by a power source in the medical device 200 itself. In some cases, there are two pairs of sensors within the plug 210 configured to respectively detect the peeling directions of the two electrode pads 208.

According to some examples, the accessory device or the medical device 200 outputs an indication of the peeling direction. In some cases, the accessory device or the medical device 200 outputs an indication of whether the detected peeling direction matches the predetermined peeling direction, or is a different peeling direction. For example, accessory device or the medical device 200 may output an indication instructing the user to check surfaces of either or both of the electrode pads 208 for the substrate, in the event that an improper peeling direction is detected. In some examples, the accessory device or the medical device 200 outputs a communication signal, to an external device, including the indication. In some cases, the accessory device or the medical device 200 includes an output device configured to output a signal with the indication directly to a user.

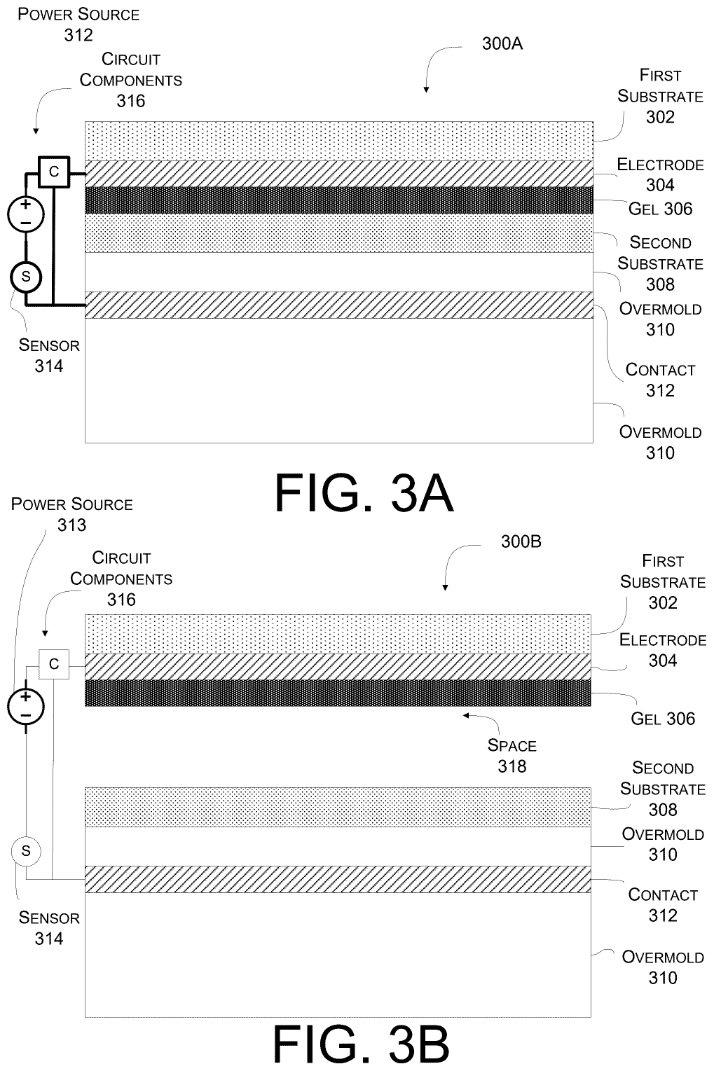

FIGS. 3A and 3B illustrate a capacitor indicative of a readiness and status of an accessory device. FIG. 3A illustrates the capacitor 300A in a first state. For instance, the capacitor 300A may be disposed in factory-sealed packaging prior to use. In various cases, an electrode pad includes a first substrate 302, an electrode 304, and a gel 306. The first substrate 302, for instance, may include an electrically insulative material (e.g., a polymer foam) that serves as a backing of the electrode pad. Although not specifically illustrated in FIG. 3A, a border region of the first substrate 302 may be covered with a biocompatible adhesive configured to attach the electrode pad to skin of a patient. The electrode 304, in various cases, is configured to receive an electrical signal (e.g., an electrical signal indicative of an ECG) from the patient and/or to output an electrical signal (e.g., an electrical shock that may be part of a defibrillation therapy) to the patient. The gel 306 facilitates an electrical connection between the electrode 304 and the skin of the patient. For instance, the gel 306 includes an electrically conductive hydrogel. In various cases, the gel 306 is biocompatible.

During storage, the electrode pad is disposed in packaging that includes a second substrate 308. According to various cases, the second substrate 308 is disposed on the gel 306 and may prevent damage to the gel 306 during storage. In various examples, the second substrate 308 prevents water from evaporating from the gel 306. For example, the second substrate 308 includes a paper and/or polymer film that is impermeable to fluids, such as water vapor.

The electrode pad, in various cases, is electrically coupled to a plug that includes an overmold 310 as well as a contact 312. For example, the overmold 310 includes a polymer that encloses other components of the plug, such as electrically conductive portions, switches, diodes, or any other electrical components. The contact 312, in various cases, is electrically conductive. For example, the contact 312 may include a conductive trace disposed inside of the plug.

In various implementations, a capacitor includes plates that include the electrode 304 and the contact 312. The gel 306, in various cases, serves as a dielectric material disposed between the electrode 304 and the contact 312. According to various implementations, a circuit includes the capacitor. For example, the circuit further includes a power source 313, a sensor 314, and additional circuit components 316. The power source 313, in various implementations, includes a battery, mains power, a capacitor, an antenna, or the like. In various cases, the power source 313 includes an antenna that induces a current in the circuit in response to receiving an electromagnetic signal from an external device. The sensor 314, in various cases, detects an electrical signal through the circuit. For example, the sensor 314 may include a voltmeter, an ammeter, or the like. The circuit components 316, in various implementations, include one or more transistors, one or more resistors, one or more capacitors, one or more inductors, one or more diodes, one or more attenuators, one or more transducers, one or more light sources, or any combination thereof. In various cases, the circuit components 316 include a wire that connects the plug to the electrode 304 in the electrode pad. The circuit components 316 may include multiple components connected to each other in series and/or in parallel. The power source 313, the sensor 314, the additional circuit components 316, or any combination thereof, are disposed inside of the plug.

The sensor 314, in various implementations, is configured to detect an electrical signal that is indicative of a capacitance of the capacitor. In some cases, the sensor 314 detects a current through the circuit and/or a voltage across one or more components of the circuit. The following Equation 2 can be used to calculate the current flowing through the circuit:

$$i = C \frac{dV}{dt} \qquad ( 2 )$$

wherein i is the current into the capacitor, C is the capacitance of the capacitor, Vis the voltage stored in the capacitor, and tis time. Depending on the circuit components 316 and the type of the sensor 314, the capacitance of the capacitor may therefore be derived based on measurements of a voltage and/or current within the circuit made by the sensor 314.

In various implementations, a readiness of the electrode pad can be determined based on the capacitance of the capacitor. For example, as provided in Equation 1, the capacitance of the capacitor is dependent on a relative permittivity of the materials between the plates of the capacitor. In particular, the relative permittivity of the gel 306 is dependent on a hydration of the gel 306. For example, if the gel 306 is hydrated, the capacitance of the capacitor may be below a threshold capacitance. However, if the gel 306 has lost a significant amount of hydration due to drying, the capacitance of the capacitor may be above the threshold capacitance. In various cases, if the gel 306 is sufficiently dried out, the electrode pad may be unsuitable for use. That is, the hydration level of the gel 306 is indicative of the readiness of the electrode pad.

FIG. 3B illustrates the capacitor 300B in a second state. In the second state, the electrode pad has been removed from the second substrate 308, such that a space 318 is disposed between the gel 306 and the second substrate 308. The presence of the space 318 also impacts the capacitance of the capacitor that includes the electrode 304, the gel 306, and the contact 312. For instance, the space 318 may include air, which may have a different relative permittivity than the gel 306. In addition, the space 318 increases the distance between the electrode 304 and the contact 312, which further impacts the capacitance of the capacitor. In various implementations, the sensor 314 may detect when the space 318 is present between the electrode 304 and the contact 312 by determining when the capacitance of the capacitor by comparing the capacitance to a threshold.

In some implementations, multiple sensors are used to determine the capacitance of capacitors defined between the electrode 304 and multiple contacts (e.g., including the contact 312) in the plug. By determining, and comparing, the different times (e.g., as evidenced by timestamps) at which the space 318 is detected between the respective capacitors, the sensors may determine a direction from which the electrode pad is peeled from packaging that includes the second substrate 308.

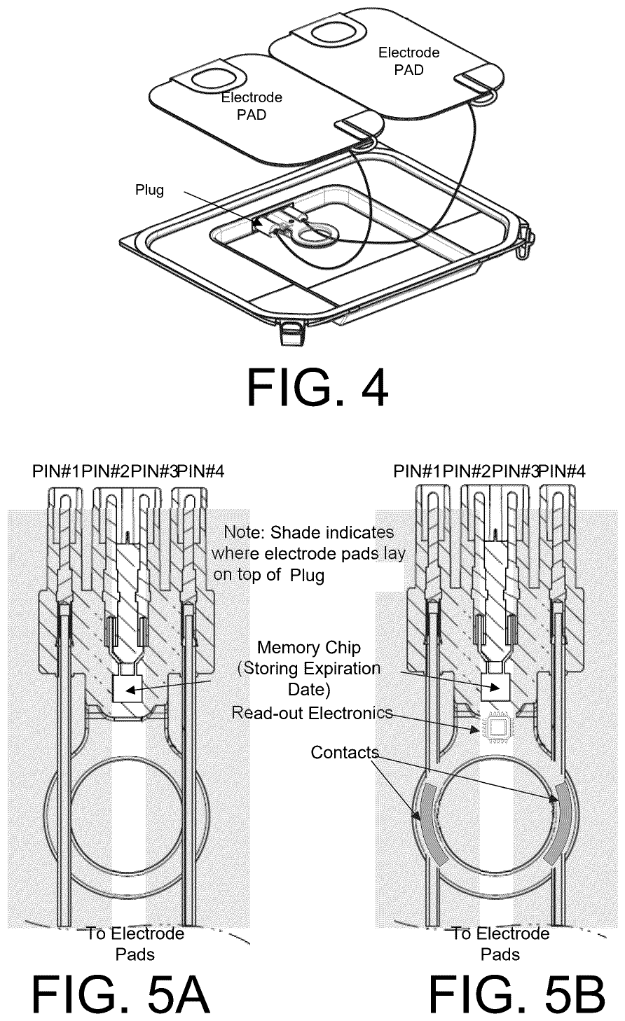

FIG. 4 illustrates a specific example of electrode pads packaged with a plug that enables self-testing. As shown, the electrode pads are connected to a plug (e.g., a QUIK-COMBO™ plug from Stryker Corporation of Kalamazoo, MI). The plug may have a loop that contains an electrically conductive trace configured to form at least one capacitor with electrodes in each of the electrode pads. During storage, the plug is disposed in a position that is adjacent to the electrode pads, such that the capacitance of each capacitor can be detected by a sensor within the package. For example, the sensor is integrated into the plug itself. By detecting a change in the capacitance of each capacitor, or by determining whether the capacitance in each capacitor is above a threshold, the sensor may detect whether the electrode pads (and the plug) are ready for use or expired. Further, the sensor may detect a peeling direction from which the electrode pads are removed from the packaging.

FIGS. 5A and 5B illustrate examples of plugs of accessory devices that can be identified using an external device. Shaded regions of FIGS. 5A and 5B indicate overlapping positions of electrode pads when the plugs and the electrode pads are disposed in packaging for storage. The electrode pads are electrically coupled to the plugs via wires (not illustrated).

According to some examples, the identifier and/or expiration date stored in the memory chip may be wirelessly read by an external device. For example, the memory chip may include an antenna that serves as an NFC tag storing the identifier and/or expiration date. Accordingly, the external device may identify the accessory device prior to use and/or may determine whether the accessory device is suitable for use with a patient, or whether it should be replaced.

FIG. 5B illustrates an example of a plug that includes a memory chip as well as contacts for detecting the readiness of a pair of electrode pads packaged with the plug. In various cases, one of the contacts overlaps a first electrode pad and the other one of the contacts overlaps a second electrode pad. According to various cases, the contacts include conductive traces that form capacitors with electrodes in the electrode pads. Read-out electronics, also disposed in the plug, are configured to determine the capacitances of the capacitors based on electrical signals received by the contacts. Further, the read-out electronics are configured to compare the capacitances to thresholds. Based on the comparisons, the read-out electronics may determine whether either of the electrode pads is expired. For example, the read-out electronics may determine that a gel disposed on the electrode of one of the electrode pads has been dried out based on determining that the capacitance of a capacitor including one of the contacts, the electrode, and the gel is greater than a threshold capacitance. In various cases, the memory chip illustrated in FIG. 5B may operate similarly to the memory chip illustrated in FIG. 5A. In some cases, the plug can further output an indication of the readiness of the electrode pads. For example, the plug may output an indication that one or both of the electrode pads is expired. In various examples, an antenna in the plug (for instance, part of the memory chip) outputs a signal indicative of the readiness of one or both of the electrode pads.

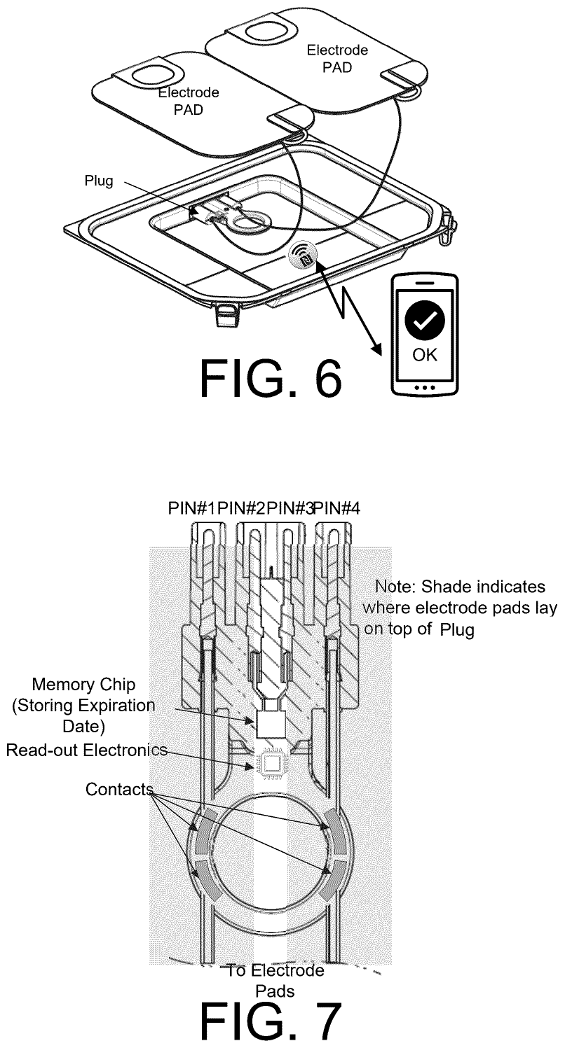

FIG. 6 illustrates examples of an electrode pad package that can report results of a self-test to an external device. As shown, the electrode pads are connected to a plug (e.g., a QUIK-COMBO plug from Stryker Corporation of Kalamazoo, MI). The plug may have a loop that contains an electrically conductive trace configured to form at least one capacitor with electrodes in each of the electrode pads. Further, the electrode pad package may include an antenna configured to output, to an external device (e.g., a mobile device, such as a tablet computer or smartphone) a communication signal that is indicative of a readiness of one or both of the electrode pads. In some cases, the antenna is disposed within the plug. For instance, the communication signal may be any wireless communication signal known in the art, such as an NFC signal, a BLUETOOTH™ signal, or the like.

FIG. 7 illustrates an example of a plug that can detect peel direction of electrode pads that are electrically connected to the plug. Shaded regions of FIG. 7 indicate overlapping positions of electrode pads when the plug and the electrode pads are disposed in packaging for storage. The electrode pads are electrically coupled to the plug via wires (not illustrated).

The plug illustrated in FIG. 7 includes a memory chip as well as contacts for detecting the readiness of a pair of electrode pads packaged with the plug.

In various cases, the plug includes two sets of contacts: one disposed under a first electrode pad and the other disposed under a second electrode pad. Each contact may form a capacitor with the electrode of the electrode pad that it overlaps.

Read-out electronics, also disposed in the plug, are configured to determine the times at which the capacitances of the capacitors change based on electrical signals received by the contacts. For instance, the read-out electronics are configured to determine when each capacitance rises above a threshold. A sudden increase in the capacitance of a capacitor indicates that the portion of the electrode pad that is part of the capacitor has been separated from the corresponding contact (see, e.g., FIG. 3B). By determining when each capacitor has a sudden increase in capacitance, a direction from which each electrode pad has been removed from the packaging can be determined. In some cases, the read-out electronics determines whether the detected peeling direction matches a predetermined peeling direction.

In various cases, a memory chip illustrated in FIG. 7 may operate similarly to the memory chip illustrated in FIG. 5A. In some cases, the plug can further output an indication of the readiness of the electrode pads. For example, the plug may output an indication that one or both of the electrode pads is expired. In various examples, an antenna in the plug (for instance, part of the memory chip) outputs a signal indicative of the readiness of one or both of the electrode pads.

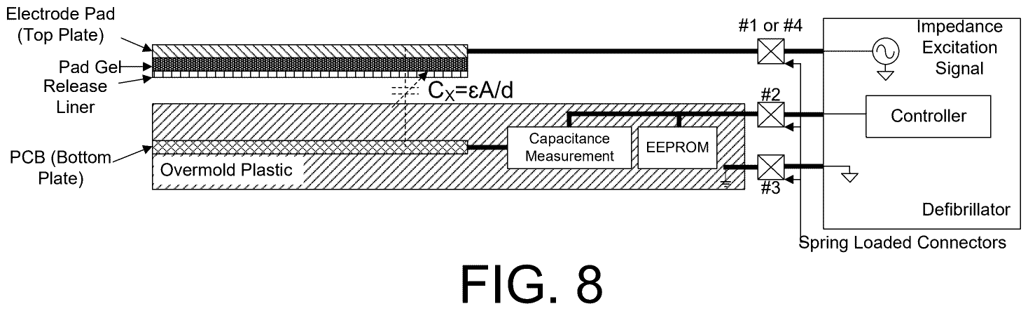

FIG. 8 illustrates a cross-section of an example electrode pad with a PCB plug that can be used to detect a readiness state of the electrode pad.

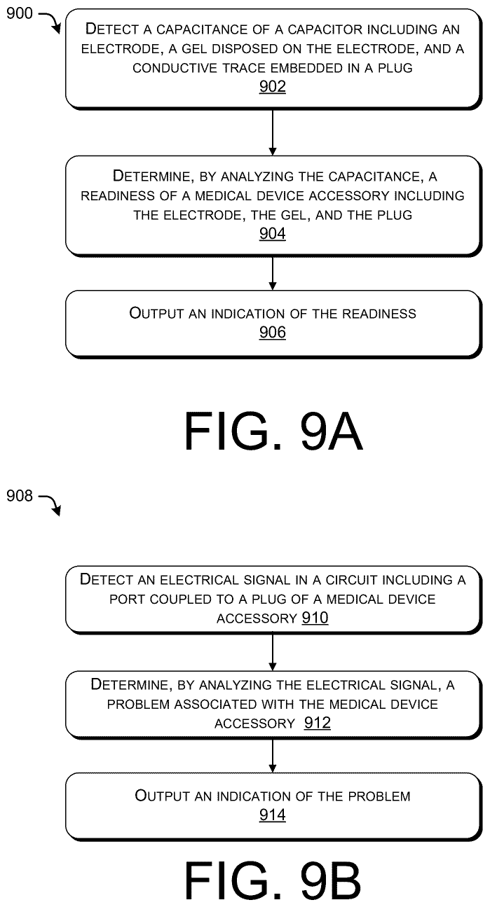

FIG. 9A illustrates an example process 900 for determining a readiness of a medical device accessory. The process 900 is performed by an entity including, for instance, an accessory device (e.g., an accessory device including the first electrode pad 104, the second electrode pad 106, and the plug 108), a sensor in the accessory device (e.g., the sensor 112), a standalone medical device (e.g., the defibrillator 110), a processor, or any combination thereof.

At 902, the entity detects a capacitance of a capacitor including an electrode, a gel disposed on the electrode, and a conductive trace embedded in a plug. In various implementations, an accessory device includes the electrode, the gel, and the plug. For example, the accessory device includes an electrode pad configured to be externally applied to the skin of a subject. In various cases, the plug is configured to be (e.g., physically and/or electrically) coupled to a medical device, such as a monitor-defibrillator or an AED.

The capacitance is detected by a sensor, for instance. In some cases, the sensor is in the medical device. In some examples, the sensor is within the accessory device itself. According to some cases, the sensor is powered by a power source of the medical device, such as a battery. In some examples, the sensor is powered by a battery included in the accessory device. According to some examples, the sensor is powered wirelessly, such as via an antenna through which a current is induced when the antenna receives an electromagnetic signal from an external device. The external device, for instance, is a mobile device, such as a mobile phone, tablet computer, or a portable medical device (e.g., a monitor-defibrillator). The electromagnetic signal, for instance, includes a BLUETOOTH™ or NFC signal. In some cases, the capacitance can be detected by the sensor before the accessory device is removed from packaging and/or coupled to the medical device.

At 904, the entity determines, by analyzing the capacitance, a readiness of a medical device accessory including the electrode, the gel, and the plug. In various cases, the capacitance is dependent on a relative permittivity of the gel. The relative permittivity, for instance, is indicative of a hydration of the gel. Therefore, the capacitance, in various cases, is dependent on whether the gel is dried out or intact. In various cases, the entity determines that the medical device accessory is expired if the capacitance is over a threshold capacitance. In some examples, the entity determines that the medical device accessory is ready-for-use if the capacitance is less than the threshold capacitance.

At 906, the entity outputs an indication of the readiness. For instance, the entity indicates the capacitance and/or whether the medical device accessory is expired or ready-for-use. In various cases, the entity outputs the indication via an output device, such as a light, a screen, a speaker, or a transceiver. In some examples, the entity transmits a communication signal encoded with the indication.

FIG. 9B illustrates an example process 908 for identifying a problem associated with a medical device accessory. The process 908 is performed by an entity including, for instance, an accessory device (e.g., an accessory device including the first electrode pad 104, the second electrode pad 106, and the plug 108), a sensor in the accessory device (e.g., the sensor 112), a standalone medical device (e.g., the defibrillator 110), a processor, or any combination thereof.

At 910, the entity detects an electrical signal in a circuit including a receptacle (e.g., a port) coupled to a plug of a medical device accessory. In various implementations, the port is removably coupled to the plug. The plug, in some examples, is electrically and physically coupled to the port. The port, for instance, is part of a medical device, such as a monitor-defibrillator or AED. In some implementations, the medical device accessory is configured to detect a physiological parameter of a subject. For instance, the medical device accessory is configured to detect an ECG, an EEG, an EOG, or an EMG of the subject. In some implementations, the medical device accessory includes one or more electrode pads configured to be externally applied to a subject's skin. In various cases, the electrical signal includes a voltage and/or current. According to some examples, the electrical signal is detected before the medical device accessory is applied to the subject.

At 912, the entity determines, by analyzing the electrical signal, a problem associated with the medical device accessory. According to some examples, the electrical signal is indicative of at least one capacitance of at least one capacitor. For example, each capacitor may include a conductive trace in the plug, an electrode, and a gel disposed on the surface of the electrode. In some cases, the problem relates to whether the medical device accessory is usable or expired. For instance, the capacitance may be indicative of a hydration of the gel. If the gel is sufficiently dried, the medical device accessory is expired. In various cases, the entity determines that the medical device accessory is usable if the capacitance is less than a threshold capacitance, or determines that the medical device accessory is expired if the capacitance is greater than the threshold capacitance. According to some examples, the entity determines times at which multiple capacitors increase in capacitance, and determines a peeling direction of the medical device accessory based on the times. The entity, for instance, may determine that a problem exists if the peeling direction is an incorrect peeling direction. In some implementations, the problem is a disconnection between the medical device and the medical device accessory. For example, if a current through the port is equal to zero, then the entity may determine that the medical device accessory is disconnected from the medical device.

At 914, the entity outputs an indication of the problem. For instance, the entity indicates the capacitance, whether the medical device accessory is expired, whether the medical device accessory has been peeled in an incorrect peeling direction, whether the medical device accessory is disconnected from the medical device, or a combination thereof. In various cases, the entity outputs the indication via an output device, such as a light, a screen, a speaker, or a transceiver. In some examples, the entity transmits a communication signal encoded with the indication.

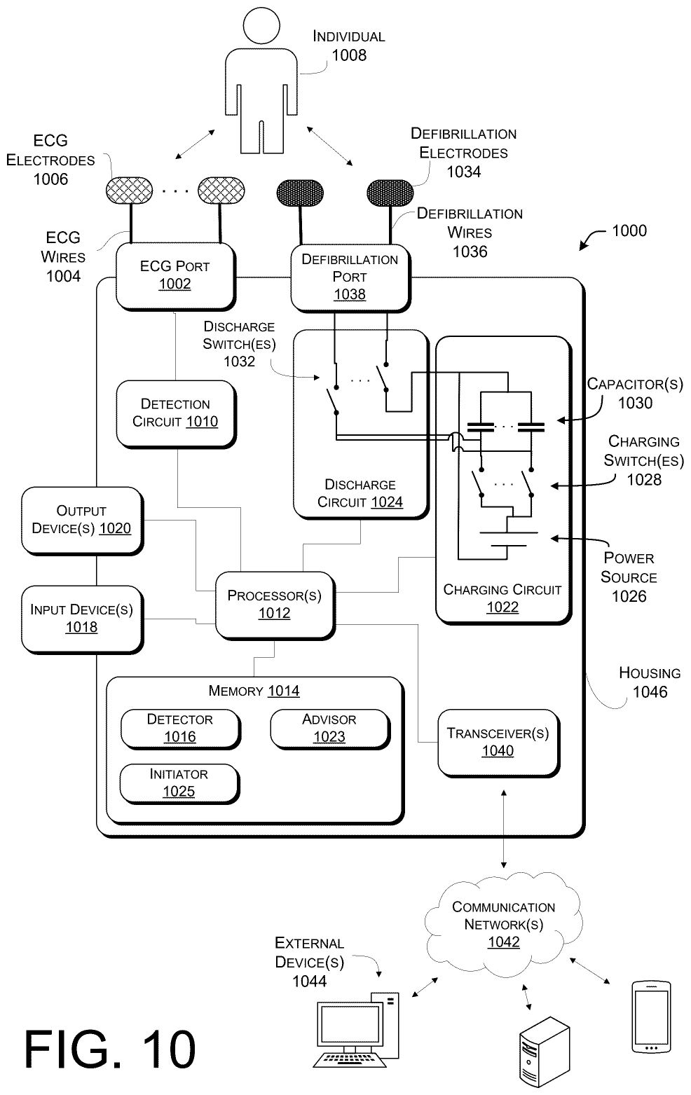

FIG. 10 illustrates an example of an external defibrillator 1100 configured to perform various functions described herein. For example, the external defibrillator 1100 is the defibrillator 110 described above with reference to FIG. 1.

The external defibrillator 1100 includes an electrocardiogram (ECG) port 1002 connected to multiple ECG wires 1004. In some cases, the ECG wires 1004 are removeable from the ECG port 1002. For instance, the ECG wires 1004 are plugged into the ECG port 1002. The ECG wires 1004 are connected to ECG electrodes 1006, respectively. In various implementations, the ECG electrodes 1006 are disposed on different locations on an individual 1008. A detection circuit 1010 is configured to detect relative voltages between the ECG electrodes 1006. These voltages are indicative of the electrical activity of the heart of the individual 1008.

In various implementations, the ECG electrodes 1006 are in contact with the different locations on the skin of the individual 1008. In some examples, a first one of the ECG electrodes 1006 is placed on the skin between the heart and right arm of the individual 1008, a second one of the ECG electrodes 1006 is placed on the skin between the heart and left arm of the individual 1008, and a third one of the ECG electrodes 1006 is placed on the skin between the heart and a leg (either the left leg or the right leg) of the individual 1008. In these examples, the detection circuit 1010 is configured to measure the relative voltages between the first, second, and third ECG electrodes 1006. Respective pairings of the ECG electrodes 1006 are referred to as “leads,” and the voltages between the pairs of ECG electrodes 1006 are known as “lead voltages.” In some examples, more than three ECG electrodes 1006 are included, such that 5-lead or 12-lead ECG signals are detected by the detection circuit 1010.

The detection circuit 1010 includes at least one analog circuit, at least one digital circuit, or a combination thereof. The detection circuit 1010 receives the analog electrical signals from the ECG electrodes 1006, via the ECG port 1002 and the ECG wires 1004. In some cases, the detection circuit 1010 includes one or more analog filters configured to filter noise and/or artifact from the electrical signals. The detection circuit 1010 includes an analog-to-digital converter (ADC) in various examples. The detection circuit 1010 generates a digital signal indicative of the analog electrical signals from the ECG electrodes 1006. This digital signal can be referred to as an “ECG signal” or an “ECG.”

In some cases, the detection circuit 1010 further detects an electrical impedance between at least one pair of the ECG electrodes 1006. For example, the detection circuit 1010 includes, or otherwise controls, a power source that applies a known voltage (or current) across a pair of the ECG electrodes 1006 and detects a resultant current (or voltage) between the pair of the ECG electrodes 1006. The impedance is generated based on the applied signal (voltage or current) and the resultant signal (current or voltage). In various cases, the impedance corresponds to respiration of the individual 1008, chest compressions performed on the individual 1008, and other physiological states of the individual 1008. In various examples, the detection circuit 1010 includes one or more analog filters configured to filter noise and/or artifact from the resultant signal. The detection circuit 1010 generates a digital signal indicative of the impedance using an ADC. This digital signal can be referred to as an “impedance signal” or an “impedance.”

The detection circuit 1010 provides the ECG signal and/or the impedance signal one or more processors 1012 in the external defibrillator 1100. In some implementations, the processor(s) 1012 includes a central processing unit (CPU), a graphics processing unit (GPU), both CPU and GPU, or other processing unit or component known in the art.

The processor(s) 1012 is operably connected to memory 1014. In various implementations, the memory 1014 is volatile (such as random access memory (RAM)), non-volatile (such as read only memory (ROM), flash memory, etc.) or some combination of the two. The memory 1014 stores instructions that, when executed by the processor(s) 1012, causes the processor(s) 1012 to perform various operations. In various examples, the memory 1014 stores methods, threads, processes, applications, objects, modules, any other sort of executable instruction, or a combination thereof. In some cases, the memory 1014 stores files, databases, or a combination thereof. In some examples, the memory 1014 includes, but is not limited to, RAM, ROM, electrically erasable programmable read-only memory (EEPROM), flash memory, or any other memory technology. In some examples, the memory 1014 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 1012 and/or the external defibrillator 1100. In some cases, the memory 1014 at least temporarily stores the ECG signal and/or the impedance signal.

In various examples, the memory 1014 includes a detector 1016, which causes the processor(s) 1012 to determine, based on the ECG signal and/or the impedance signal, whether the individual 1008 is exhibiting a particular heart rhythm. For instance, the processor(s) 1012 determines whether the individual 1008 is experiencing a shockable rhythm that is treatable by defibrillation. Examples of shockable rhythms include ventricular fibrillation (VF) and ventricular tachycardia (V-Tach). In some examples, the processor(s) 1012 determines whether any of a variety of different rhythms (e.g., asystole, sinus rhythm, atrial fibrillation (AF), etc.) are present in the ECG signal.

The processor(s) 1012 is operably connected to one or more input devices 1018 and one or more output devices 1020. Collectively, the input device(s) 1018 and the output device(s) 1020 function as an interface between a user and the defibrillator 1100. The input device(s) 1018 is configured to receive an input from a user and includes at least one of a keypad, a cursor control, a touch-sensitive display, a voice input device (e.g., a microphone), a haptic feedback device (e.g., a motor, gyroscope, etc.), or any combination thereof. The output device(s) 1020 includes at least one of a display, a speaker, a haptic output device, a printer, or any combination thereof. In various examples, the processor(s) 1012 causes a display among the input device(s) 1018 to visually output a waveform of the ECG signal and/or the impedance signal. In some implementations, the input device(s) 1018 includes one or more touch sensors, the output device(s) 1020 includes a display screen, and the touch sensor(s) are integrated with the display screen. Thus, in some cases, the external defibrillator 1100 includes a touchscreen configured to receive user input signal(s) and visually output physiological parameters, such as the ECG signal and/or the impedance signal.

In some examples, the memory 1014 includes an advisor 1023, which, when executed by the processor(s) 1012, causes the processor(s) 1012 to generate advice and/or control the output device(s) 1020 to output the advice to a user (e.g., a rescuer). In some examples, the processor(s) 1012 provides, or causes the output device(s) 1020 to provide, an instruction to perform cardiopulmonary resuscitation (CPR) on the individual 1008. In some cases, the processor(s) 1012 evaluates, based on the ECG signal, the impedance signal, or other physiological parameters, CPR being performed on the individual 1008 and causes the output device(s) 1020 to provide feedback about the CPR in the instruction. According to some examples, the processor(s) 1012, upon identifying that a shockable rhythm is present in the ECG signal, causes the output device(s) 1020 to output an instruction and/or recommendation to administer a defibrillation shock to the individual 1008.

The memory 1014 also includes an initiator 1025 which, when executed by the processor(s) 1012, causes the processor(s) 1012 to control other elements of the external defibrillator 1100 in order to administer a defibrillation shock to the individual 1008. In some examples, the processor(s) 1012 executing the initiator 1025 selectively causes the administration of the defibrillation shock based on determining that the individual 1008 is exhibiting the shockable rhythm and/or based on an input from a user (received, e.g., by the input device(s) 1018. In some cases, the processor(s) 1012 causes the defibrillation shock to be output at a particular time, which is determined by the processor(s) 1012 based on the ECG signal and/or the impedance signal.

The processor(s) 1012 is operably connected to a charging circuit 1022 and a discharge circuit 1024. In various implementations, the charging circuit 1022 includes a power source 1026, one or more charging switches 1028, and one or more capacitors 1030. The power source 1026 includes, for instance, a battery. The processor(s) 1012 initiates a defibrillation shock by causing the power source 1026 to charge at least one capacitor among the capacitor(s) 1030. For example, the processor(s) 1012 activates at least one of the charging switch(es) 1028 in the charging circuit 1022 to complete a first circuit connecting the power source 1026 and the capacitor to be charged. Then, the processor(s) 1012 causes the discharge circuit 1024 to discharge energy stored in the charged capacitor across a pair of defibrillation electrodes 1034, which are in contact with the individual 1008. For example, the processor(s) 1012 deactivates the charging switch(es) 1028 completing the first circuit between the capacitor(s) 1030 and the power source 1026, and activates one or more discharge switches 1032 completing a second circuit connecting the charged capacitor 1030 and at least a portion of the individual 1008 disposed between defibrillation electrodes 1034.

The energy is discharged from the defibrillation electrodes 1034 in the form of a defibrillation shock. For example, the defibrillation electrodes 1034 are connected to the skin of the individual 1008 and located at positions on different sides of the heart of the individual 1008, such that the defibrillation shock is applied across the heart of the individual 1008. The defibrillation shock, in various examples, depolarizes a significant number of heart cells in a short amount of time. The defibrillation shock, for example, interrupts the propagation of the shockable rhythm (e.g., VF or V-Tach) through the heart. In some examples, the defibrillation shock is 200 J or greater with a duration of about 0.015 seconds. In some cases, the defibrillation shock has a multiphasic (e.g., biphasic) waveform. The discharge switch(es) 1032 are controlled by the processor(s) 1012, for example. In various implementations, the defibrillation electrodes 1034 are connected to defibrillation wires 1036. The defibrillation wires 1036 are connected to a defibrillation port 1038, in implementations. According to various examples, the defibrillation wires 1036 are removable from the defibrillation port 1038. For example, the defibrillation wires 1036 are plugged into the defibrillation port 1038.

Although not specifically illustrated in FIG. 10, in some examples, the ECG electrodes 1006 and the defibrillation electrodes 1034, as well as the ECG wires 1004 and the defibrillation wires 1036, are integrated into an accessory device. In some examples, electrode pads include the ECG electrodes 1006 and the defibrillation electrodes 1034. In some cases, wires including the ECG wires 1004 and/or the defibrillation wires 1036 are electrically coupled to a plug that is configured to be inserted into the ECG port 1002 and/or the defibrillation port 1038. For instance, the plug includes one or more sensors configured to detect a readiness of the accessory device.

In various implementations, the processor(s) 1012 is operably connected to one or more transceivers 1040 that transmit and/or receive data over one or more communication networks 1042. The transceiver(s) 1040 include at least one transmitter and/or at least one receiver. For example, the transceiver(s) 1040 includes a network interface card (NIC), a network adapter, a local area network (LAN) adapter, or a physical, virtual, or logical address to connect to the various external devices and/or systems. In various examples, the transceiver(s) 1040 includes any sort of wireless transceivers capable of engaging in wireless communication (e.g., radio frequency (RF) communication). For example, the communication network(s) 1042 includes one or more wireless networks that include a 3rd Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 1040 includes other wireless modems, such as a modem for engaging in WI-FI®, WIGIG®, WIMAX®, BLUETOOTH®, or infrared communication over the communication network(s) 1042.

The defibrillator 1100 is configured to transmit and/or receive data (e.g., ECG data, impedance data, data indicative of one or more detected heart rhythms of the individual 1008, data indicative of one or more defibrillation shocks administered to the individual 1008, etc.) with one or more external devices 1044 via the communication network(s) 1042. The external devices 1044 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 1042. In some examples, the external device(s) 1044 is located remotely from the defibrillator 1100, such as at a remote clinical environment (e.g., a hospital). According to various implementations, the processor(s) 1012 causes the transceiver(s) 1040 to transmit data to the external device(s) 1044. In some cases, the transceiver(s) 1040 receives data from the external device(s) 1044 and the transceiver(s) 1040 provide the received data to the processor(s) 1012 for further analysis.

In various implementations, the external defibrillator 1100 also includes a housing 1046 that at least partially encloses other elements of the external defibrillator 1100. For example, the housing 1046 encloses the detection circuit 1010, the processor(s) 1012, the memory 1014, the charging circuit 1022, the transceiver(s) 1040, or any combination thereof. In some cases, the input device(s) 1018 and output device(s) 1020 extend from an interior space at least partially surrounded by the housing 1046 through a wall of the housing 1046. In various examples, the housing 1046 acts as a barrier to moisture, electrical interference, and/or dust, thereby protecting various components in the external defibrillator 1100 from damage.

In some implementations, the external defibrillator 1100 is an automated external defibrillator (AED) operated by an untrained user (e.g., a bystander, layperson, etc.) and can be operated in an automatic mode. In automatic mode, the processor(s) 1012 automatically identifies a rhythm in the ECG signal, makes a decision whether to administer a defibrillation shock, charges the capacitor(s) 1030, discharges the capacitor(s) 1030, or any combination thereof. In some cases, the processor(s) 1012 controls the output device(s) 1020 to output (e.g., display) a simplified user interface to the untrained user. For example, the processor(s) 1012 refrains from causing the output device(s) 1020 to display a waveform of the ECG signal and/or the impedance signal to the untrained user, in order to simplify operation of the external defibrillator 1100.

In some examples, the external defibrillator 1100 is a monitor-defibrillator utilized by a trained user (e.g., a clinician, an emergency responder, etc.) and can be operated in a manual mode or the automatic mode. When the external defibrillator 1100 operates in manual mode, the processor(s) 1012 cause the output device(s) 1020 to display a variety of information that may be relevant to the trained user, such as waveforms indicating the ECG data and/or impedance data, notifications about detected heart rhythms, and the like.

### Example Clauses

The following example clauses provide various implementations of the present disclosure. However, the scope of the present disclosure is not limited to the example clauses provided.
1. An electrode package, including: a plug configured to be coupled to an external defibrillator; an electrode; a gel coated on the electrode and including a dielectric material; a conductive trace embedded in the plug; a sensor configured to detect a capacitance of a capacitor including the electrode, the gel, and the conductive trace; an output device configured to output an indication of readiness of the electrode corresponding to the capacitance of the capacitor; an electrically insulative substrate disposed on the electrode, the electrode being disposed between the electrically insulative substrate and the gel; and a housing enclosing the plug, the electrode, the electrically insulative substrate, and the gel, the housing being configured to hold the plug in a position such that the gel is disposed between the electrode and the sensor.
2. The electrode package of clause 1, further including: an antenna electrically coupled to the sensor, a current being induced in the sensor in response to the antenna receiving an electromagnetic signal from an external transmitter, wherein the sensor is embedded in the plug.
3. The electrode package of clause 1 or 2, wherein the sensor is included in the external defibrillator.
4. A medical device accessory, including: a plug configured to be coupled to a medical device; an electrode; a gel coated on the electrode and including a dielectric material; and a conductive trace embedded in the plug, wherein a sensor is configured to identify a readiness of the medical device accessory by detecting a capacitance of a capacitor including the electrode, the gel, and the conductive trace.
5. The medical device accessory of clause 4, wherein the gel includes a hydrogel that includes an electrolyte.
6. The medical device accessory of clause 5, wherein the readiness of the medical device accessory is indicative of a hydration of the hydrogel.
7. The medical device accessory of any of clauses 4 to 6, wherein the sensor is included in the medical device.
8. The medical device accessory of any of clauses 4 to 7, further including: the sensor embedded in the plug.
9. The medical device accessory of clause 8, further including: an antenna configured to induce a current in the sensor in response to receiving an electromagnetic signal from an external device.
10. The medical device accessory of clause 9, wherein a frequency of the electromagnetic signal is in a range of 10 to 2500 MHz.
11. The medical device accessory of any of clauses 4 to 10, further including: an output device configured to output an indication of the readiness of the medical device accessory.
12. The medical device accessory of clause 11, wherein the output device includes a light, a screen, a speaker, or a transceiver.
13. A method, including: detecting a capacitance of a capacitor including an electrode, a gel disposed on the electrode, and a conductive trace embedded in a plug, the plug being configured to couple to a medical device; determining, by analyzing the capacitance, a readiness of a medical device accessory including the electrode, the gel, and the plug; and outputting an indication of the readiness.
14. The method of clause 13, wherein detecting the capacitance is performed by a sensor included in the medical device.
15. The method of clause 13 or 14, wherein detecting the capacitance is performed by a sensor included in the plug.
16. The method of clause 15, further including: receiving, by an antenna, an electromagnetic signal from an external device; and in response to receiving the electromagnetic signal, activating a sensor, wherein detecting the capacitance of the capacitor is performed by the sensor.
17. The method of any of clauses 13 to 16, wherein determining, by analyzing the capacitance, the readiness of the medical device accessory includes: determining that the capacitance is above a threshold, and wherein outputting the indication of the readiness includes outputting an indication that the medical device accessory is expired.
18. The method of any of clauses 13 to 17, wherein determining, by analyzing the capacitance, the readiness of the medical device accessory includes: determining that the capacitance is below a threshold, and wherein outputting the indication of the readiness includes outputting an indication that the medical device is usable.
19. The method of any of clauses 13 to 18, wherein outputting the indication of the readiness includes transmitting a communication signal including the indication of the readiness.
20. The method of any of clauses 13 to 19, further including: in response to outputting the indication of the readiness, using the medical device accessory to detect a physiological parameter of a subject or to administer a treatment to the subject.
21. A system, including: a medical device accessory including: electrodes coated with a gel and configured to be adhered to a chest of a subject; a plug including a conductive trace; and wires electrically coupling the electrodes to the plug; and an automated external defibrillator (AED), including: a measurement circuit electrically coupled to the plug, the measurement circuit configured to detect an electrical signal from a circuit including an example electrode among the electrodes, the gel, the conductive trace, and an example wire among the wires, the electrical signal being indicative of a capacitance of a capacitor including the example electrode, the gel, and the conductive trace; an output device; and a processor configured to: determine that the capacitance is above a threshold; and in response to determining that the capacitance is above the threshold, cause the output device to output an instruction to replace the medical device accessory.
22. The system of clause 21, wherein the measurement circuit is configured to detect the electrical signal from the circuit every 24 hours.
23. The system of clause 21 or 22, wherein the output device includes: a display configured to visually present the instruction to replace the medical device accessory; a speaker configured to audibly output the instruction to replace the medical device accessory; or a transceiver configured to transmit, to an external device, a communication signal encoding the instruction to replace the medical device accessory.
24. A medical device, including: a measurement circuit electrically coupled to a medical device accessory and configured to detect an electrical signal indicative of a capacitance of a capacitor in the medical device accessory, the capacitor including an electrode, a gel coating the electrode, and a conductive trace; and a processor configured to: determine, by analyzing the capacitance, whether the medical device accessory is usable or expired; and output an indication of whether the medical device accessory is usable or expired.
25. The medical device of clause 24, wherein the measurement circuit is further configured to detect a physiological parameter of a subject connected to the medical device accessory, wherein the processor is configured to determine that the accessory is unexpired, and wherein the processor is further configured to, in response to determining that the accessory is unexpired, cause the measurement circuit to detect the physiological parameter of the subject coupled to the medical device accessory.
26. The medical device of clause 25, wherein the physiological parameter includes an electrocardiogram (ECG), an electroencephalogram (EEG), an electrooculogram (EOG), or an electromyograph (EMG).
27. The medical device of any of clauses 24 to 26, wherein the measurement circuit is electrically coupled to the medical device accessory by a plug including the conductive trace.
28. The medical device of any of clauses 24 to 27, wherein the processor is configured to determine, by analyzing the capacitance, whether the medical device accessory is expired by comparing the capacitance to a threshold.
29. The medical device of any of clauses 24 to 28, wherein the processor is configured to output the indication of whether the medical device accessory is expired by outputting an instruction to replace the medical device accessory.
30. The medical device of any of clauses 24 to 29, further including: an output device configured to output the indication of whether the medical device accessory is expired.
31. The medical device of any of clauses 24 to 30, further including: a treatment circuit configured to output, to the medical device accessory, a treatment signal, wherein the processor is configured to determine that the medical device accessory is unexpired, and wherein the processor is further configured to: in response to determining that the medical device accessory is unexpired, cause the treatment circuit to output, to the medical device accessory, the treatment signal.
32. The medical device of any of clauses 24 to 31, the electrical signal being a first electrical signal, the capacitor being a first capacitor, the conductive trace being a first conductive trace, wherein the measurement circuit is further configured to detect a second electrical signal indicative of a capacitance of a second capacitor in the medical device accessory, the second capacitor including the electrode, the gel coating the electrode, and a second conductive trace, and wherein the processor is further configured to: determine, by analyzing the first capacitance and the second capacitance, a direction at which a peel is removed from the electrode; determine that the direction at which the peel is removed from the electrode is a predetermined direction; and in response to determining that the direction at which the peel is removed from the electrode is the predetermined direction, output an instruction to check the electrode.
33. A method, including: detecting, by a medical device, an electrical signal in a circuit including a port coupled to a plug of a medical device accessory; determining, by analyzing the electrical signal, a problem associated with the medical device accessory; and outputting an instruction to correct the problem.
34. The method of clause 33, wherein the circuit includes a capacitor, the capacitor including an electrode of the medical device and a gel disposed on the electrode, and wherein the electrical signal is indicative of a capacitance of the capacitor.
35. The method of clause 34, wherein determining, by analyzing the electrical signal, the problem associated with the medical device accessory includes: determining that the medical device accessory is expired by determining that the capacitance of the capacitor is greater than a predetermined threshold, and wherein outputting the instruction to correct the problem includes outputting an instruction to replace the medical device accessory.
36. The method of any of clauses 34 to 35, the electrical signal being a first electrical signal, the circuit being a first circuit, the capacitor being a first capacitor, the method further including: detecting, by the medical device, a second electrical signal indicative of a capacitance of a second capacitor, the second capacitor including the electrode of the medical device and the gel, wherein determining the problem associated with the medical device accessory includes: detecting a change in the first capacitance; detecting a change in the second capacitance; identifying a direction from which a peel is removed from the electrode and the gel by analyzing a time of the change in the first capacitance and a time of the change in the second capacitance; and determining that the direction from which the peel is removed from the electrode and the gel is a predetermined direction associated with incorrectly removing the peel.
37. The method of any of clauses 33 to 36, wherein determining, by analyzing the electrical signal, the problem associated with the medical device accessory includes: determining that the circuit is incomplete, wherein outputting the instruction to correct the problem includes an instruction to re-connect the medical device accessory to the medical device.
38. The method of any of clauses 33 to 37, further including: determining that the problem has been corrected; and in response to determining that the problem has been corrected, detecting, using the medical device accessory or a replacement medical device accessory, a physiological parameter of a subject.
39. The method of any of clauses 33 to 38, further including: determining that the problem has been corrected; and in response to determining that the problem has been corrected, outputting, using the medical device accessory or a replacement medical device accessory, a treatment to a subject.
40. The method of any of clauses 33 to 39, wherein detecting, by the medical device, the electrical signal in the circuit is performed at a predetermined frequency.

### CONCLUSION

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. An electrode package, comprising:
a plug configured to be coupled to an external defibrillator;
an electrode;
a gel coated on the electrode and comprising a dielectric material;
a conductive trace embedded in the plug;
a sensor configured to detect a capacitance of a capacitor comprising the electrode, the gel, and the conductive trace;
an output device configured to output an indication of readiness of the electrode corresponding to the capacitance of the capacitor;
an electrically insulative substrate disposed on the electrode, the electrode being disposed between the electrically insulative substrate and the gel; and
a housing enclosing the plug, the electrode, the electrically insulative substrate, and the gel, the housing being configured to hold the plug in a position such that the gel is disposed between the electrode and the sensor.

2. The electrode package of claim 1, further comprising:
an antenna electrically coupled to the sensor, a current being induced in the sensor in response to the antenna receiving an electromagnetic signal from an external transmitter,
wherein the sensor is embedded in the plug.

3. The electrode package of claim 1, wherein the sensor is comprised in the external defibrillator.

4. A medical device accessory, comprising:
a plug configured to be coupled to a medical device;
an electrode;
a gel coated on the electrode and comprising a dielectric material; and
a conductive trace embedded in the plug,
wherein a sensor is configured to identify a readiness of the medical device accessory by detecting a capacitance of a capacitor comprising the electrode, the gel, and the conductive trace.

5. The medical device accessory of claim 4, wherein the gel comprises a hydrogel that comprises an electrolyte.

6. The medical device accessory of claim 5, wherein the readiness of the medical device accessory is indicative of a hydration of the hydrogel.

7. The medical device accessory of claim 4, wherein the sensor is comprised in the medical device.

8. The medical device accessory of claim 4, further comprising:
the sensor embedded in the plug.

9. The medical device accessory of claim 8, further comprising:
an antenna configured to induce a current in the sensor in response to receiving an electromagnetic signal from an external device.

10. The medical device accessory of claim 9, wherein a frequency of the electromagnetic signal is in a range of 10 to 2500 MHz.

11. The medical device accessory of claim 4, further comprising:
an output device configured to output an indication of the readiness of the medical device accessory.

12. The medical device accessory of claim 11, wherein the output device comprises a light, a screen, a speaker, or a transceiver.

13. A method, comprising:
detecting a capacitance of a capacitor comprising an electrode, a gel disposed on the electrode, and a conductive trace embedded in a plug, the plug being configured to couple to a medical device;
determining, by analyzing the capacitance, a readiness of a medical device accessory comprising the electrode, the gel, and the plug; and
outputting an indication of the readiness.

14. The method of claim 13, wherein detecting the capacitance is performed by a sensor comprised in the medical device.

15. The method of claim 13, wherein detecting the capacitance is performed by a sensor comprised in the plug.

16. The method of claim 15, further comprising:
receiving, by an antenna, an electromagnetic signal from an external device; and
in response to receiving the electromagnetic signal, activating a sensor,
wherein detecting the capacitance of the capacitor is performed by the sensor.

17. The method of claim 13, wherein determining, by analyzing the capacitance, the readiness of the medical device accessory comprises:
determining that the capacitance is above a threshold, and
wherein outputting the indication of the readiness comprises outputting an indication that the medical device accessory is expired.

18. The method of claim 13, wherein determining, by analyzing the capacitance, the readiness of the medical device accessory comprises:
determining that the capacitance is below a threshold, and
wherein outputting the indication of the readiness comprises outputting an indication that the medical device is usable.

19. The method of claim 13, wherein outputting the indication of the readiness comprises transmitting a communication signal comprising the indication of the readiness.

20. The method of claim 13, further comprising:
in response to outputting the indication of the readiness, using the medical device accessory to detect a physiological parameter of a subject or to administer a treatment to the subject.
